(19) United States
(12) Patent Application Publication
(10) Pub. No.: US 2018/0000508 A1
(43) Pub. Date: Jan. 4, 2018

(54) SCALPEL FOR PERFORMING A CESAREAN SECTION

(71) Applicant: JMD Innovation Inc., Burnaby (CA)

(72) Inventors: Lina HU, Burnaby (CA); Huafeng LI, Burnaby (CA); Ji ZHANG, Burnaby (CA)

(21) Appl. No.: 15/520,819

(22) PCT Filed: Oct. 22, 2015
(86) PCT No.: PCT/CA2015/051071

§ 371 Date: Apr. 20, 2017

Related U.S. Application Data
(60) Provisional application No. 62/067,015, filed on Oct. 22, 2014.

(51) Int. Cl. A61B 17/3209; A61B 17/3211; A61B 17/42

(52) U.S. Cl. CPC A61B 17/32093; A61B 17/3211; A61B 17/42

(57) ABSTRACT

A surgical scalpel including a handle with a proximal end and a distal end and which is hollow defines a notch that encases a blade. The scalpel alternatively may include two blades, wherein the blade which is used to perform the initial is retracted during the remainder of the procedure.

### CROSS-REFERENCE TO RELATED APPLICATIONS

This application claims the benefit of U.S. Provisional Application No. 62/067,015, filed Oct. 22, 2014, the contents of which are incorporated herein by reference in their entirety.

### TECHNICAL FIELD

The subject matter described herein relates to medical instruments and methods and pare particularly to the scalpels, and methods, used in performing the surgical operation cesarean section and other surgical procedures.

### BACKGROUND

A cesarean section is a major surgical procedure in which a baby is removed from the uterus by making a cut into the abdomen and then into the uterus. In many cases a cesarean section is necessary to save the life of the baby or the mother. In other cases a cesarean section is performed when a vaginal birth is not possible, e.g., failure of normal progression of labor. In some other cases a cesarean section may be scheduled due to a patient's request, or recommended by another doctor.

There are a number of potential complications that may occur as a result of a cesarean section. The complications include, but are not limited to, infection, bleeding, traumatic injury and death. It is estimated that the chance of dying from a cesarean section, which is a major abdominal surgery, is about 0.02% (20 out of 100,000), which is higher than the chance of dying from vaginal delivery. The reason cesarean sections may involve so many complications is because they involve opening up the abdomen and making an incision into the uterus. When doing a cesarean section an obstetrician/gynecologist (ob/gyn) doctor first makes sure that the patient has adequate anesthesia. They commonly use epidural or spinal anesthesia. On unusual occasions the patient may need to have general anesthesia, which involves putting the patient completely to sleep with a tube in the throat and the use of a ventilator device. Once the patient is anesthetized, a transverse incision about the size of the distance from the wrist to the tip of the ring finger is made into the skin, using a sharp scalpel. In most cases this incision is made from side-to-side, just above the pubic hair line (sometimes called a “bikini cut”). In other countries, and in some emergency situations, an up-and-down cut is made below the belly button to the top of the bikini line. Most doctors prefer bikini cuts because they heal and look better, and cause less pain after leaving the hospital. After cutting through the skin and underlying fat cells, the doctor will make a transverse incision, using a sharp scalpel, through the remaining tissue and then will enter the abdominal cavity. The bladder, uterus, ovaries, tubes and intestines are all visible. The vesicouterine fold is opened and the bladder is retracted. The uterus is then cut. When the uterus is cut the amniotic fluid will flow out, although in some cases there is only a small amount. Some doctors will then enlarge the cut in the uterus using their fingers. An alternative for lengthening the incision is to use a special scissor. After entering the uterus through the cut, the baby is carefully grasped, and the surgical assistant pushes on the top of the uterus to deliver the baby through the hole in the uterus.

Cesarean sections are a major surgery and can have many complications. Some complications that can occur, during or after a cesarean section, include heavy bleeding which may require blood transfusions, damage to the bladder or intestines, damage to blood vessels, infections of the uterus, kidneys, lungs or other areas, opening up of the skin incision, blood clots around the uterus or in the leg veins or lungs, and an inability of the blood to clot. On rare occasions, a hysterectomy may be performed to save the mother's life.

One of the most troubling adverse risks of cesarean sections is the possibility of cutting or nicking the baby while it is in the uterus, causing it to bleed. In some cesarean sections, and at some times, there is very little amniotic fluid to protect the baby. The baby, due to its position, may have a part of its body directly in contact with the inner wall of the uterus at the position where the doctor makes the incision through the uterus. The sharp scalpel may cut or nick the baby, causing an accidental laceration (cut). The baby's laceration, which is unexpected, may lead to extensive scarring and disfigurement. Such scarring and disfigurement may cause permanent or long-term damage, for example, amputation of a finger. Laceration of the nose, eyes, mouth or internal organs may be difficult or impossible to repair.

The cesarean section begins with an incision or cut on the skin. This cut is carried deeper until the abdomen is completely open (into the peritoneal cavity). The bladder, which is normally attached to the front of the uterus, is released. This is done by cutting the attachments of the bladder to the uterus and pushing it away. A cut is then made in the uterus. This cut is then carried deeper until the uterine wall is completely divided. The uterine incision is then extended by tearing the tissue or cutting it with a sharp scissor. The amniotic cavity, a baby sac with its surrounding fluid (“waters”) is opened. The baby is then delivered and handed to the pediatric or baby care team. The after-birth, or placenta, is removed. The incision is closed and the abdominal wall is reapproximated.

Several points should be emphasized. The uterine wall can vary greatly in thickness, due to individual variation, prior surgery, the result of labor, and other factors. The amount of amniotic fluid present, that normally cushions the baby, can also vary markedly (even be depleted), especially following rupture of the membrane. These factors underscore the difficulty in creating the incision and may predispose a cesarean section to complications, involving both the mother and the baby.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

The present disclosure provides an improvement in the surgical technique of the cesarean section, or other surgery, using a special curved, blunt-edge scalpel which encases a sharp-edged blade. Although the description is primarily in terms of cesarean section, the blunt-edge instruments may be used with other surgical procedures.

The device disclosed herein consists of a body which is sized and shaped so that it can be held securely and comfortably by a physician. It includes a body forming a curved anterior portion which encases a blade which is used to make an initial puncture in the skin or other membrane. The anterior portion is then inserted into the resulting hole and is advanced, causing the blade to make an incision of a desired length. As the incision is made, a bottom surface of the anterior portion ensures an adequate separation between the cutting blade and the underlying tissue, thereby preventing undesirable damage to the physician, the mother or the fetus.

In one embodiment, the anterior portion of the body comprises a slot having a narrow open mouth. In another embodiment, the body encases the blade wherein a portion of the blade can extend across the slot such that the cutting edge of the blade is exposed. In one embodiment, the blade is movable such that different portions of the cutting edge may be exposed across the slot. Preferably the blade is enclosed within the main body save for the portion thereof which extends across the slot.

In one aspect, the body encases a first and a second blade, wherein the first blade as located anterior to the second blade and wherein the second blade can extend across the notch to expose the cutting edge of the second blade. In another embodiment, the first blade is exposed when the initial cut of the skin is performed. In still another embodiment, the first blade is exposed only during the initial cut of the skin is performed.

In one embodiment, the first blade is retractable.

In one embodiment, the first and/or second blade is slidable along the slot in the main body.

In one embodiment, the device comprises a spring means, which may be a compression spring or a tension spring, being connected between one end of the first and/or second blade and a fixed part of the handle. For example, the spring means may be encased in the handle.

In one embodiment, the exposed portion of the cutting edge of the first and/or second blade may extend at an acute angle to the longitudinal surface of the tapered tip portion. In this case the material to be cut slides up the surface of the tapered tip portion and enters the acute angle between the blade and that surface with a wedging action, leading to the severing of the material.

In any of the above arrangements the side of the slot forming an acute angle with the cutting edge of the blade may be formed with a longitudinal groove or gap into which the cutting edge of the blade partly extends. In use the material (for example, skin) to be cut becomes partly forced into the groove or gap causing it to be bent over the cutting edge of the blade, which is found to enhance the cutting action.

In one embodiment, the body encases a single blade which comprises an anterior portion and a posterior portion. In another embodiment, the single blade can be rotated to move the anterior and/or posterior portion of the single blade from an exposed position to an encased position. In yet another embodiment, the single blade extends across the notch to expose the cutting edge of the second blade.

In one embodiment, the anterior portion of the single blade is exposed when an initial cut of the skin is performed. In another embodiment, the anterior portion of the single blade is exposed only during the initial cut of the skin is performed.

In another aspect, a method for performing a cesarean section is provided comprising use of a device as described herein.

Although the devices as disclosed herein may be used in various surgical procedures, its use will be described in detail only in connection with an improved cesarean section surgical procedure.

### BRIEF DESCRIPTION OF DRAWINGS

FIG. 1 is a diagram of a device comprising a first and a second blade according to the present disclosure.

FIGS. 2A-2B are diagrams of a device comprising a blade having an anterior and posterior portion according to the present disclosure.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

The first portion of the cesarean section procedure of the present invention is conventional. First, the patient is partially, or fully, anesthetized. Then a transverse incision (cut) is made in the abdomen using a sharp scalpel. For example, the incision may be the so-called bikini cut. In all cases, the incision is through the wall of the abdomen. The vesicouterine fold is opened and the bladder is retracted. Up to this point the surgical operation has followed the conventional and well-established procedure for cesarean sections.

In addition to the possibility of accidental laceration of the baby, the use of a traditional sharp scalpel also has the risk of the doctor extending the transverse incision too far and cutting blood vessels or muscle structure on the sides of the uterus. The use of the device of the present disclosure avoids that risk. Although it will separate the uterine fibers, when used properly it is unable to to sever blood vessels or muscle structure.

The baby's body is, at times, directly flush with the inner wall of the uterus. During many cesarean sections the amniotic fluid cushion is absent. That cushion of fluid normally protects the baby from surgical trauma. Such trauma may ensue following the contact if a sharp blade scalpel should penetrate the uterine wall and accidentally come in contact with the baby's body surface, causing a laceration.

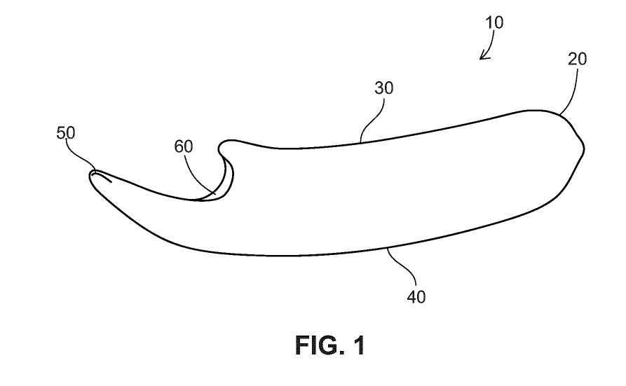

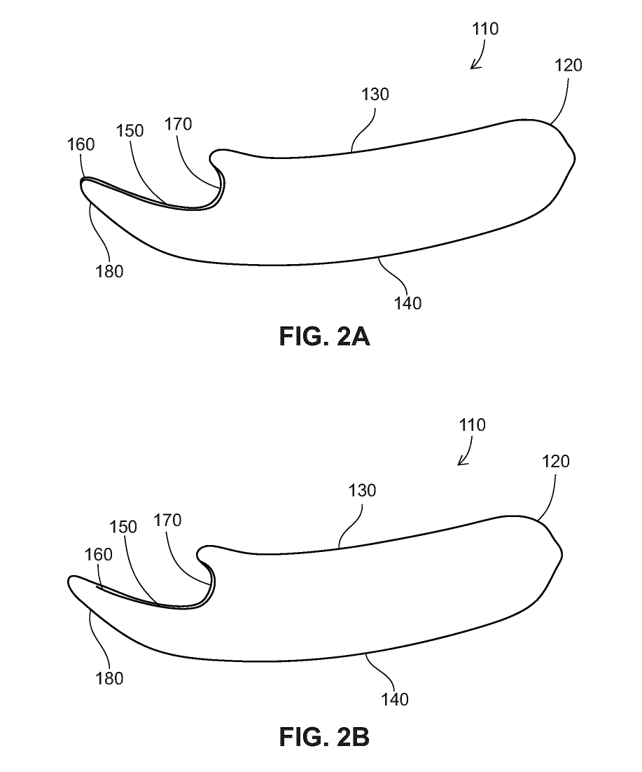

FIGS. 1 and 2 show devices 10 and 110 according to the present disclosure. Each consists of a handle or body (20 or 120, respectively), which can be made of metal or plastic. If device 10 or 110 is made of metallic material, it can be sterilizable and reusable. In an alternative embodiment, handle 10 is disposable and made of, for example, plastic. Handle 20 has a top edge 30 and a bottom edge 40, wherein top edge 30 and bottom edge 40 are curved to provide the scalpel with a body that is easy to grasp. As illustrated at least in FIG. 2A, handle 120 has a top edge 130 and a bottom edge 140, wherein top edge 130 and bottom edge 140 are curved to provide the scalpel with a body that is easy to grasp. The curvature of the edges also can provide the physician with a clear indication of the correct orientation for the scalpel during surgery. Importantly, each device 10 and 110 has a notch (shown as 70 in device 10 and 170 in device 110) located between an anterior portion (80 in device 10 and 180 in device 110) and a ledge portion (90 in device 10 and 195 in device 110). Each notch houses the cutting edge of a blade as described below, providing protection from accidental or unintentional cutting by a practitioner with the device.

Device 10 comprises a first blade 50 and a second blade 60. First blade 50 is encased in part by the anterior portion 80 of handle 20. Device 10 can be disposable or resusable. When device 10 is disposable, it is generally used only for a single operation, since it may become dull or nicked. Each of first blade 50 and second blade 60 has a razor-sharp edge. That edge is sufficiently sharp so that, with very little pressure, it will cut normal skin. For example, if lightly drawn across a finger, it will cut the skin and the finger will bleed. Generally the blades are available in stainless steel or carbon steel and may be individually wrapped to protect their sterility.

In one embodiment, first blade 50 has a pointed tip to facilitate an initial incision of the skin. In another embodiment, second blade 60 has a concave portion as depicted, for example, in FIG. 1.

When device 10 is first contacted with the skin, first blade 50 is exposed to complete an initial cut. Importantly, the sharp cutting edge of first blade 50 is positioned such that it is oriented away from tissues (fetus) present beneath the initial cut. After the initial cut, device 10 is advanced to make a longitudinal incision in the skin. As device 10 is advanced, first blade 50 is automatically retracted. Second blade 60 can be exposed or sheathed during the initial cut by first blade 50. Second blade 60 is exposed as device 10 is advanced to cut longitudinally through the skin and, optionally, underlying tissue.

A second aspect of the device is illustrated in FIGS. 2A and 2B as device 110. As shown in FIG. 2A, device 110 encases a single blade 150 shaped such that two portions, represented in FIG. 1 as anterior blade portion 160 and posterior blade portion 170, may be exposed or sheathed within a handle 120 either together or independently. Blade 150 comprises an exterior edge which comprises a sharp cutting edge which may run along the partial or full length of the exterior edge of blade 150. As with device 10, handle 120 of device 110 has a top edge 130 and a bottom edge 140.

In one embodiment, single blade 150 has a pointed tip to facilitate an initial incision of the skin. In another embodiment, posterior blade portion 170 has a concave portion as depicted, for example, in FIG. 2A.

When device 110 is first contacted with the skin in preparation for puncture or incision, anterior portion 160 of single blade 150 is exposed to complete an initial cut. Importantly, the sharp cutting edge of blade 150 is positioned such that it is oriented away from tissues (fetus) present beneath the initial cut. After the initial cut, device 110 is advanced to make a longitudinal incision in the skin. As device 110 is advanced, single blade 150 rotates slightly to result in anterior portion 160 of single blade 150 being encased within the anterior portion of handle 120. FIG. 2B illustrates this embodiment, showing that the anterior end of single blade 150 has retracted in a posterior direction and thus anterior portion 160 of single blade 150 is now fully encased within anterior portion 180 of device 110. Posterior portion 170 of single blade 150 becomes exposed with the rotation of single blade 150 which resulted in sheathing of anterior portion 160.

Device 10 or 110 is molded from a plastic or metal material to have a hollow portion which can encase partially or wholly first blade 50 and/or second blade 60 of device 10 or anterior section 160 and/or posterior section 170 of device 110. This hollow portion can be straight, convex or concave.

Preferably, the body of device 10 or 110 has an overall length of about 3 to 5 inches, and more particularly between 4 to 6 or 4 to 5 inches, because range can accommodate physicians with hands in the range of 6 to 8 inches. The width of the body of device 10 or 110 can range between 0.8 to 1.0 inches and have a thickness of between 0.150 and 0.250 inches. A scalpel having a body of about 4.8 inches in length, a width at point 80 of 0.9 inches and a maximum thickness of about 0.2 inches is particularly advantageous.

In one aspect is a method for performing a cesarean section procedure using the device as disclosed herein. The patient is partially or fully anesthetized. A transverse incision (cut) is made through the wall of the abdomen using a sharp scalpel. Then, device 10 or device 110 is used to cut through the wall of the uterus. An incision is made using first blade 50 of device 10 or anterior portion 160 of device 110, preferably of less than 1 mm, through the tough outer surface layer of the uterus. Device 10 or 110 is then advanced to make a transverse incision, wherein the incision involves cutting by second blade 60 or posterior portion 170. This separates muscle fiber and opens up the uterus wall, forming a transverse incision. This incision is through the wall of the uterus, e.g., through the myometrium and the endometrium.

The remainder of the cesarean section operation is conventional. The incision may, if desired, be enlarged by hand pressure. The baby is extracted and the uterus and abdomen repaired.

In addition to the possibility of accidental laceration of the baby, the use of a sharp scalpel also has the risk of the doctor extending the transverse incision too far and cutting blood vessels or muscle structure on the sides of the uterus. The use of the blunt scalpel of the present invention avoids that risk. Although it will separate the uterine fibers, it is too blunt to sever blood vessels or muscle structure.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. (canceled)

2. (canceled)

3. (canceled)

4. (canceled)

5. (canceled)

6. A device for performing a cesarean section, the device comprising:
an elongate body comprising a curved anterior portion, a notch positioned proximal to the curved anterior portion, and a posterior portion, proximal to the curved anterior portion;
a retractable blade component encased within the elongate body, the blade component comprising first and second blades, the first blade being encased within the curved anterior portion, the second blade being encased within the body, adjacent to the notch, the second blade being positionable across the notch;
wherein the curved anterior portion is movable relative to the first blade to expose the first blade from the curved anterior portion to make an initial puncture in a uterine wall, the curved anterior portion further being moveable relative to the first blade to retract the first blade into the curved anterior portion after the initial puncture of the uterine wall to permit insertion of the curved anterior portion into the puncture, and
wherein a bottom surface of the curved anterior portion is spaced apart from the second blade to separate the second blade from underlying tissue to prevent contact between the second blade and the underlying tissue.

7. The device of claim 6, wherein the anterior portion of the body comprises a slot, the first blade extending across the slot when exposed.

8. The device of claim 7, wherein the first blade is movable to different positions within the slot to permit different portions of a cutting edge of the first blade to be exposed across the slot.

9. The device of claim 6, wherein the first blade is located anterior to the second blade and wherein the second blade extends across the notch to expose a cutting edge of the second blade.

10. The device of claim 6, wherein the first blade is exposed before contact between the uterine wall and the device.

11. The device of claim 6, wherein the first blade is exposed only during the initial puncture of the uterine wall is performed.

12. The device of claim 7, wherein the first and second blade are slidable along the slot of the body.

13. The device of claim 6, further comprising a spring means connected between an end of the first blade and a fixed part of the elongate body.

14. The device of claim 6, further comprising a spring means connected between an end of the second blade and a fixed part of the elongate body.

15. The device of claim 6, wherein the first blade and the second blade are separate.

16. The device of claim 6, wherein the first blade and the second blade move relative to each other during use.

17. The device of claim 6, wherein the second blade has a concave portion.

18. The device of claim 6, wherein after the initial puncture, first blade is automatically retracted.

19. The device of claim 6, wherein the retractable blade component is a single, continuous blade in which the first blade is an anterior blade portion and the second blade is a posterior blade portion.

20. The device of claim 19, wherein the anterior blade portion and the posterior blade portion are exposed or sheathed within the elongate body independently of each other.

21. The device of claim 19, wherein the posterior blade portion of the single blade has a concave portion.

22. The device of claim 19, wherein the single blade is rotatable relative to the body to result in the anterior blade portion being sheathed within the curved anterior portion.

23. The device of claim 22, wherein when the anterior blade portion retracts within the curved anterior portion upon rotation of the single blade, the posterior blade portion becomes exposed.

24. A method of performing a caesarean section using a blunt scalpel device for preventing trauma to a fetus and mother, the method comprising:
contacting a blunt anterior housing of the blunt scalpel device against a uterine wall;
piercing the uterine wall by exerting pressure to expose a first blade component of the device from the blunt anterior housing;
after the piercing, retracting the first blade component into the blunt anterior housing; and
advancing the device to make a longitudinal incision in the uterine wall using a second blade component.

25. The method of claim 24, wherein the first blade is automatically retracted into the blunt anterior housing.

26. The method of claim 24, wherein the second blade component is sheathed during the piercing, the second blade component being exposed as device is advanced to cut longitudinally through the uterine wall.

27. The method of claim 24, wherein the advancing comprises separating uterine fibers without severing blood vessels or muscle structure. A device for performing a cesarean section, the device comprising: [symbol]
